(19) United States
(12) Patent Application Publication
(10) Pub. No.: US 2010/0138192 A1
(43) Pub. Date: Jun. 3, 2010

(54) Systems and Methods for Selecting Components for Use in RF Filters Within Implantable Medical Device Leads Based on Inductance, Parasitic Capacitance and Parasitic Resistance

(75) Inventor: Xiaoyi Min, Thousand Oaks, CA (US)

Correspondence Address: PACESETTER, INC., 15900 VALLEY VIEW COURT, SYLMAR, CA, 91392-9221, US

(73) Assignee: PACESETTER, INC., Sylmar, CA (US)

(21) Appl. No.: 12/325,945

(22) Filed: Dec. 1, 2008

(51) Int. Cl. G06F 17/50; A61N 1/05

(52) U.S. Cl. 703/1; 607/116

(57) ABSTRACT

Techniques are provided for selecting and configuring inductors for use in radio-frequency (RF) inductive filters within pacing/sensing leads of pacemakers or implantable cardioverter-defibrillators. The filters are employed to reduce heating due to induced currents caused by magnetic resonance imaging (MRI) procedures or other sources of strong RF fields. In particular, techniques are provided for determining optimal inductance values by taking into account parasitic resistances and parasitic capacitances of the inductors. Tolerances of the inductive devices are also taken into account.

### CROSS REFERENCE TO RELATED APPLICATIONS

This application is related to copending U.S. patent application Ser. No. 11/955,268, filed Dec. 12, 2007, titled “Systems and Methods for Determining Inductance and Capacitance Values for Use with LC Filters Within Implantable Medical Device Leads to Reduce Lead Heating During an MRI” (Attorney Docket A07P1182).

### FIELD OF THE INVENTION

The invention generally relates to leads for use with implantable medical devices, such as pacemakers or implantable cardioverter-defibrillators (ICDs) and, in particular, to systems and methods for determining inductance values for use in inductive (L) filtering elements (including self-resonant inductors or LC resonators) within such leads to reduce tip heating during magnetic resonance imaging (MRI) procedures.

### BACKGROUND OF THE INVENTION

MRI is an effective, non-invasive magnetic imaging technique for generating sharp images of the internal anatomy of the human body, which provides an efficient means for diagnosing disorders such as neurological and cardiac abnormalities and for spotting tumors and the like. Briefly, the patient is placed within the center of a large superconducting magnetic that generates a powerful static magnetic field. The static magnetic field causes protons within tissues of the body to align with an axis of the static field. A pulsed radio-frequency (RF) magnetic field is then applied causing the protons to begin to precess around the axis of the static field. Pulsed gradient magnetic fields are then applied to cause the protons within selected locations of the body to emit RF signals, which are detected by sensors of the MRI system. Based on the RF signals emitted by the protons, the MRI system then generates a precise image of the selected locations of the body, typically image slices of organs of interest.

However, MRI procedures are problematic for patients with implantable medical devices such as pacemakers and ICDs. One of the significant problems or risks is that the strong RF fields of the MRI can induce currents through the lead system of the implantable device into the tissues resulting in Joule heating in the cardiac tissues around the electrodes of leads, potentially damaging adjacent tissues. Indeed, in worst-case scenarios, the temperature at the tip of an implanted lead has been found to increase as much as 70 degrees Celsius (C) during an MRI tested in a gel phantom in a non-clinical configuration. Although such a dramatic increase is probably unlikely within a clinical system wherein leads are properly implanted, even a temperature increase of only about 6°-13° C. might cause myocardial tissue damage.

Furthermore, any significant heating of cardiac tissues near lead electrodes can affect the pacing and sensing parameters associated with the tissues near the electrode, thus potentially preventing pacing pulses from being properly captured within the heart of the patient and/or preventing intrinsic electrical events from being properly sensed by the device. The latter might result, depending upon the circumstances, in therapy being improperly delivered or improperly withheld. Another significant concern is that any currents induced in the lead system can potentially generate voltages within cardiac tissue comparable in amplitude and duration to stimulation pulses and hence might trigger unwanted contractions of heart tissue. The rate of such contractions can be extremely high, posing significant clinical risks on patients. Therefore, there is a need to reduce heating in the leads of implantable medical devices, especially pacemakers and ICDs, and to also reduce the risks of improper tissue stimulation during an MRI, which is referred to herein as MRI-induced pacing.

A variety of techniques have been developed. See, for example, the following patents and patent applications: U.S. Pat. Nos. 6,871,091; 6,930,242; 6,944,489; 6,971,391; 6,985,775; U.S. Patent Application Nos. 2003/0083723; 2003/0083726; 2003/0144716; 2003/0144718; 2003/0144719; and 2006/0085043; as well as the following PCT documents WO 03/037424; WO 03/063946; and WO 03/063953. At least some of these techniques are directed to the use of RF filters within the leads for use in filtering RF signals induced by MRIs.

When patients with implants are scanned during an MRI procedure, the wavelength in tissue (55 cm) is comparable to lead length (46 cm to 58 cm). The conductors in the leads of the implants act like an antenna and waves can propagate along the leads sending currents into tissues through electrodes of the leads causing heating. This is referred to as RF heating. The amount of RF heating from an MRI procedure is complicated, subject to many variables including lead length, lead configuration, lead design, the MRI machine and patient anatomical variables. However, if high impedance can be created near electrodes, the current near the electrodes can be forced at or near zero. From transmission line theory, the reflection coefficient is equal to: Γ=(Z_{L}−Z0)/(Z_{L}+Z0) where Γ is reflection coefficient, Z_{L} is load and Z0 is characteristic impedance of transmission line. If Z_{L}=0, then Γ=1 and considerable RF heating can occur. However, if Z_{L}=infinite, then Γ=1 and current I=0, such that there is no RF heating. This is the basis of the band stop filter concept that can be used to limit current flow out of electrodes into tissue. Theoretically, Z_{L} can be inductive, capacitive, or resistive. Once |Z_{L}| is large enough and ratio of |Z0|/|Z_{L}| is small, Γ is close to 1 and current I is small, reducing or eliminating RF heating from the MRI. Thus, to eliminate RF heating near electrodes of implantable device leads, high impedance at RF signal frequencies of MRI is needed. In-vitro tests have shown that significant heat reduction in gel phantoms can be achieved with impedance greater than 1000 ohms.

However, issues arise in determining optimal values for L and C to achieve desired impedance for use within the RF filters mounted within device leads. Preferably, L and C values are selected so that the RF filter resonates at the frequency of RF signals induced by the MRI, such as at a resonant frequency of, e.g., about 64 MHz (i.e. in the range of 63.45-64.15 MHz) of a 1.5 tesla (T) MRI or about 128 MHz (i.e. in the range of 123-128.1 MHz) of a 3 T MRI. In principle, a wide range of L and C values can potentially be used to achieve a desired resonant frequency based on: 2π*f=1/sqrt(L*C) wherein “f” is the resonant frequency. (Solving for L, this equation may also be represented as: L=1/(4π²f² C).) However, it has been discovered that not all combinations of permissible L and C values are equally effective in reducing lead heating. Some problems arise for different pairs of L and C due to tolerances of the individual inductors and capacitors used within the RF filters. These issues were addressed in U.S. patent application Ser. No. 11/955,268, filed Dec. 12, 2007, of Min, entitled “Systems and Methods for Determining Inductance and Capacitance Values for use with LC Filters within Implantable Medical Device Leads to Reduce Lead Heating during an MRI,” which is fully incorporated by reference herein.

The present invention is directed to providing further improvements in the use of inductive elements within implantable medical device leads and, in particular, to techniques for taking parasitic capacitance and parasitic resistance into account within inductive filtering elements.

### SUMMARY OF THE INVENTION

In accordance with a first exemplary embodiment of the invention, a method is provided for designing a lead for use with an implantable medical device to achieve adequate filtering at a selected MRI RF signal frequency, wherein the lead includes an inductive filtering element to reduce lead heating due to RF fields, the inductive filtering element having an inductance (L) and a parasitic capacitance (Cs). Briefly, candidate inductive components for use as the filtering element are identified and tolerances for the inductances and the parasitic capacitances of the candidate components are input or otherwise determined. Suitable values for inductance and parasitic capacitance sufficient to achieve a target impedance value at a selected frequency (such as at about 64 MHz or at about 128 MHz) are then determined based on the tolerances for the inductances and parasitic capacitances of the candidate components. Particular components are then selected and installed for use as the inductive filtering element of the lead based, in part, on the suitable values for the parasitic capacitance and the inductance.

In this manner, the tolerances and parasitic capacitances of inductors are taken into account when designing a lead so as to achieve sufficiently high impedances at a selected frequency despite variations in inductance or parasitic capacitance due to device tolerance. In one exemplary embodiment, an inductor is selected with a self-resonant frequency (SRF) near either 64 MHz or 128 MHz so as to provide heat reduction at one of these standard MRI RF signal frequencies. (It should be understood that 64 MHz and 128 MHz are merely approximate values for the MRI frequencies. More precisely, MRIs typically operate at 63.7±0.345 MHz with 1.5 T and 125.6±3.6 MHz with 3.0 T.) Note that the SRF for the inductor arises due to the presence of inductance and parasitic capacitance within the inductor. The specific SRF value for the inductor depends on the particular values of inductance and parasitic capacitance of the inductor.

In an illustrative embodiment, a suitable target lower bound (Z₀) for the impedance is selected to achieve heat reduction during an MRI. For either 64 MHz or 128 MHz MRI RF fields, Z₀ can typically be set to 1000 ohms. Assuming low parasitic resistance (i.e. Rs<<ωL/1000 or Q_{L}>1000), suitable values for the parasitic capacitance (Cs) and the inductance (L) of the inductor are then identified by determining ranges of values that satisfy:

Cs₀<(1−ΔL /L₀)/[(ω₀Z₀(−ΔCs/Cs₀+ΔCs/Cs₀*ΔL/L₀−ΔL/L₀)]

or

L₀>Z₀*(−ΔCs/Cs₀+ΔCs/Cs₀*ΔL/L₀−ΔL/L₀)/ω₀(1−ΔL/L₀)

where Cs₀ represents a central value for the parasitic capacitance, L₀ represents a central value for the inductance, ΔCs/Cs₀ represents the tolerance of the parasitic capacitance of the inductive filtering element, ΔL/L₀ represents the tolerance of the inductance of the inductive filtering element, ω₀ represents the resonant frequency. For each Cs₀ value found to be suitable, the corresponding L₀ value may then be derived based on L₀=1/(ω₀²*Cs₀). Conversely, for each L₀ value found to be suitable, the corresponding Cs₀ may then be derived based on Cs₀=1/(ω₀²*L₀).

Once suitable ranges of values for L₀ and Cs₀ have been determined, a particular inductor is then selected from among a set of candidate inductors provided by manufactures by identifying the components having central values for parasitic capacitance and inductance close to the determined values of Cs₀ and L₀ (and which are otherwise suited for use in implantable medical leads). Parasitic resistance (Rs) can also be taken into account. Lower Rs values are associated with higher resonance Q-factor values and less component heating at resonance. Rs less than 75 ohms is generally preferred. If the parasitic resistance is not small, then, rather than using the inequalities listed above to determine ranges of suitable values for inductance and parasitic capacitance, inequalities accounting for parasitic resistance are instead used. For example, for a given L₀, a range of suitable values for Cs₀ may be determined based on:

Cs₀²<(1+Q_{L}²(1−ΔCs/Cs₀)²)/[ω₀²Z₀²*(Q_{L}²*(ΔCs/Cs₀+ΔCs/Cs₀ΔL/L₀ΔL/L₀)²+(1+ΔCs/Cs₀ )²)

where ω₀=1/sqrt(L₀Cs₀), Q_{L}=ω₀L₀/Rs and tolerance of ΔCs/Cs₀ and ΔL/L₀. This equation is derived from the more general equation for the impedance of an inductor:

Z²=(1+Q_{L}²)/[(YS−Q_{L}ωCs)²+ω²Cs²] (1)

where Ys=1/Rs, Q_{L}=ω₀L/Rs, and ω is representative of a frequency.

In accordance with a second exemplary embodiment of the invention, a method is provided for designing a lead for use with an implantable medical device to achieve adequate filtering at two or more separate MRI RF signal frequencies, wherein the lead includes an inductive filtering element to reduce lead heating due to RF fields, the inductive filtering element having an inductance (L) and a parasitic capacitance (Cs). Briefly, an SRF is selected between at least two separate MRI RF signal frequencies. A target impedance to be achieved at each of the RF signal frequencies, such as at least 1000 ohms, is also selected. Suitable values for inductance and parasitic capacitance are then determined that are sufficient to achieve the target impedance at each of the separate frequencies by, for example, selecting an inductance and then determining a range of suitable parasitic capacitance values sufficient to achieve the target impedance. Particular inductive components are then selected and installed for use in the inductive filtering element of the lead based, in part, on the selected inductance value and on the range of suitable values for the parasitic capacitance determined for that inductance value.

In this manner, the parasitic capacitances of SRF inductors can be taken into account when designing a lead so as to achieve sufficiently high impedances at both 64 MHz and 128 MHz (or at a set of three of more RF signal frequencies.) In one exemplary embodiment, an inductor is selected having an SRF in between 64 MHz and 128 MHz to provide heat reduction at both 64 MHz and 128 MHz. The inductor is selected from a set of candidate inductors based on parasitic capacitance values so as to ensure that an impedance of at least 1000 ohms is achieved at both of the RF signal frequencies. Preferably, parasitic resistance values and device tolerance values are also taken into account.

Typically, the higher the inductance, the wider the range of permissible parasitic capacitance (Cₛ) values, and hence the more likely an inductor can be found from among a group of candidate inductors that will properly filter RF signals from both 64 MHz/1.5 T and 128 MHz/3.0 T MRIs. For example, to achieve an impedance of 1000 ohms at either 64 MHz or 128 MHz using an inductor of 2.0 μH would require an inductor having a parasitic capacitance in the relatively wide range of about 0.9 to 2.0 pF (assuming negligible parasitic resistance). In contrast, to achieve the same impedance at both 64 MHz and 128 MHz using an inductor of only 1.5 μH would require that the inductor have a parasitic capacitance in the narrower range of about 1.5 pF to 2.2 pF (again assuming negligible parasitic resistance).

Hence, in one particular example, an inductor might be selected for use as an RF filter in a cardiac pacing/sensing lead that has an inductance of 2.0 μH, a parasitic capacitance (Cₛ) of 1.0 pF (to accommodate both 64 MHz and 128 MHz MRIs) and a parasitic resistance (Rₛ) of 75 ohms (to be less than the tip resistance of the lead.) Such an inductor would achieve significant reductions in tip temperatures during either 64 MHz/1.5 T or 128 MHz/3.0 T MRIs, compared to similar leads without RF filters, assuming minimal ranges of tolerances in the stated inductance.

More generally, for an SRF between 64 MHz and 128 MHz, the impedance Z needs to be greater than a targeted lower bound (Z₀) for the amplitude of the impedance (i.e. Z>Z₀ where Z₀ 1000 ohms or greater) at both 64 MHz and 128 MHz. To achieve the desired |Z₀| at both 64 MHz and 128 MHz, a selection criteria is established using Equation (1) based on |Z|>|Z₀| at 64 MHz and |Z|>|Z₀| at 128 MHz. In practice, a set of inequalities are generated (discussed below) from which ranges of suitable inductance and parasitic capacitance values are derived. The same general method can be applied for use with three or more RF signal frequencies.

The component selection and specification techniques are particularly well suited for use with RF inductive filtering components for use in cardiac pacing/sensing leads for use with pacemakers and ICDs but may also be employed in connection with other filtering components or for use in other types of leads or for use with other implantable medical devices. Also, the techniques described herein are applicable, where appropriate, to inductive-capacitive elements having both an inductor and a capacitor, as well as to inductive-capacitive elements having an inductor with parasitic capacitance. Still further, the techniques are applicable to inductive-capacitive-resistive (LCR) circuits or circuits including lumped inductors.

### BRIEF DESCRIPTION OF THE DRAWINGS

The above and further features, advantages and benefits of the invention will be apparent upon consideration of the descriptions herein taken in conjunction with the accompanying drawings, in which:

FIG. 1 is a stylized representation of an MRI system along with a patient with a pacer/ICD implanted therein with RV and LV leads employing inductive RF filters near their distal ends;

FIG. 2 is a block diagram, partly in schematic form, illustrating a bipolar lead for use with the pacer/ICD of FIG. 1 wherein an RF filter element is connected along a tip conductor to reduce tip heating during an MRI, and also illustrating a pacer/ICD connected to the lead;

FIG. 3 is a schematic diagram illustrating an equivalent circuit for the RF filter of FIG. 2, showing parasitic resistance and parasitic capacitance;

FIG. 4 is a flow diagram summarizing a method for determining suitable inductors for use with the RF filter of FIG. 2 for filtering one selected MRI RF signal frequency such as 64 Mhz or 128 Mhz while taking parasitic capacitance into account, wherein an SRF at either 64 MHz or 128 MHz is exploited;

FIG. 5 is a graph illustrating self-resonance curves for an inductor sufficient to provide a high impedance at either 64 Mhz or 128 MHz (but not both) in accordance with the general technique of FIG. 4;

FIG. 6 is a flow diagram illustrating an exemplary method for determining suitable inductance and parasitic capacitance values for use with the general “single MRI frequency” technique of FIG. 4;

FIG. 7 is a flow diagram illustrating a particular exemplary methods for determining ranges of suitable inductance values for use with the method of FIG. 6 based on tolerances in inductance and parasitic capacitance;

FIG. 8 is a flow diagram summarizing a method for determining suitable inductors for use with the RF filter of FIG. 2 for filtering two separate MRI RF signal frequencies such as filtering both 64 Mhz and 128 Mhz while taking parasitic capacitance into account, wherein an SRF between 64 MHz and 128 MHz is exploited;

FIG. 9 is a graph illustrating a self-resonance curve for an inductor sufficient to provide a high impedance at both 64 Mhz and 128 MHz for filtering both of the MRI RF signal frequencies in accordance with the general technique of FIG. 8;

FIG. 10 is a flow diagram illustrating an exemplary method for determining suitable inductance and parasitic capacitance values for use with the general technique of FIG. 9;

FIG. 11 schematic diagram illustrating an alternative circuit for the RF filter of FIG. 2, showing an LCR circuit;

FIG. 12 is a simplified, partly cutaway view, illustrating the pacer/ICD of FIG. 1 along with a more complete set of leads implanted in the heart of the patient, wherein the RV and LV leads each include an RF filtering element near tip electrodes of the leads; and

FIG. 13 is a functional block diagram of the pacer/ICD of FIG. 12, illustrating basic circuit elements that provide cardioversion, defibrillation and/or pacing stimulation in four chambers of the heart.

### DETAILED DESCRIPTION OF THE PREFERRED EMBODIMENTS

The following description includes the best mode presently contemplated for practicing the invention. The description is not to be taken in a limiting sense but is made merely to describe general principles of the invention. The scope of the invention should be ascertained with reference to the issued claims. In the description of the invention that follows, like numerals or reference designators will be used to refer to like parts or elements throughout.

### Overview of MRI System

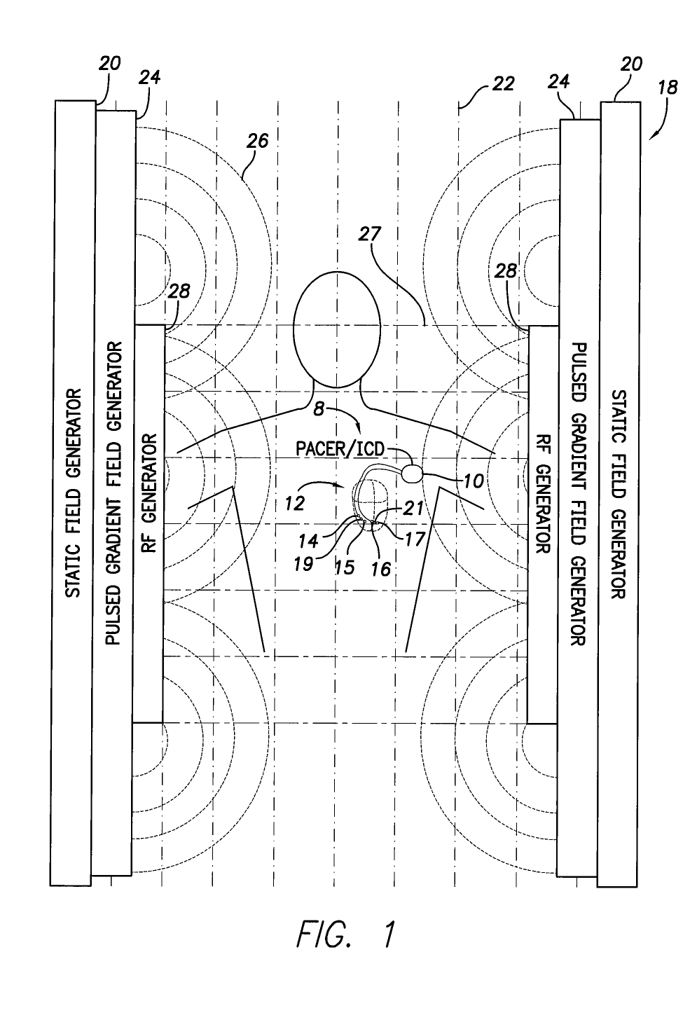

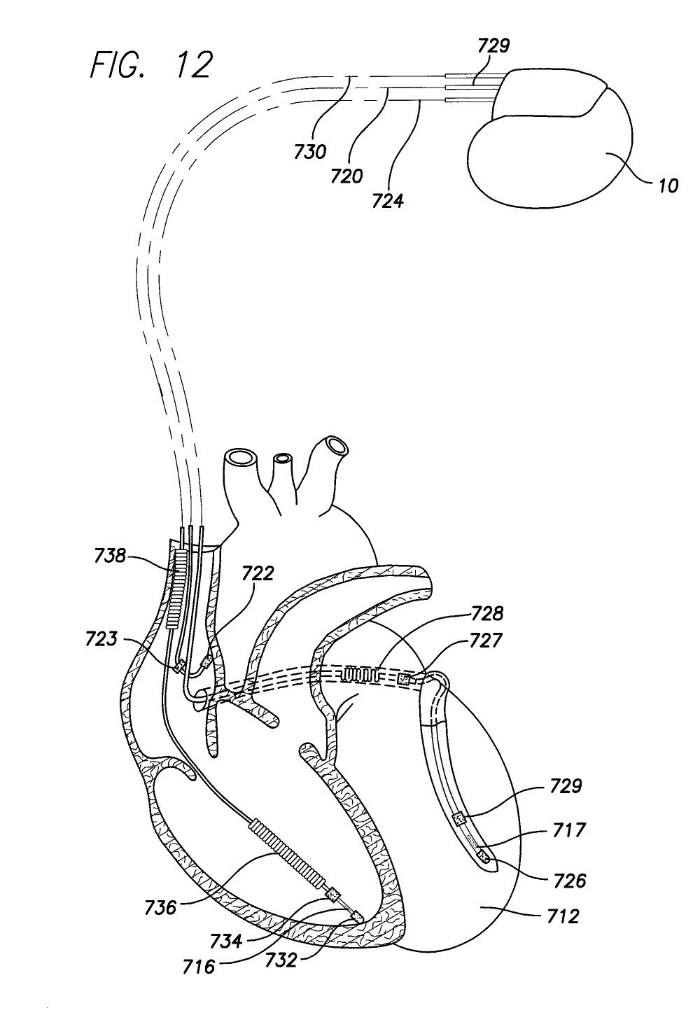

FIG. 1 illustrates an implantable medical system 8 having a pacer/ICD 10 for use with a set of coaxial bipolar pacing/sensing leads 12, which include tip and ring electrodes 14, 15, 16 and 17, as well as inductive RF filtering elements 19 and 21 (which are internal to the lead) for use in filtering RF signals. The filtering elements are connected to the tip electrodes 15, 17 of the respective leads (or to ring electrodes) so as to reduce lead heating caused by loop currents generated by an MRI system 18 to further reduce or prevent improper stimulation of the heart due to such loop currents. In FIG. 1, only two leads are shown, a right ventricular (RV) lead and a left ventricular (LV) lead. A more complete lead system is illustrated in FIG. 12, described below. As will be explained further, the inductors of the RF filtering elements are selected during lead design so as to achieve optimal filtering of current loops induced by the RF fields of an MRI or other source of RF fields. In some implementations, one or more additional leads may be provided (such as a right atrial (RA) lead). RF filtering elements may be provided within the additional leads as well. Also, in some implementations, no LV lead is provided.

As to the MRI system 18, the system includes a static field generator 20 for generating a static magnetic field 22 and a pulsed gradient field generator 24 for selectively generating pulsed gradient magnetic fields 26. The MRI system also includes an RF generator 28 for generating RF fields 27 generally at about 64 MHz or 128 MHz. (Again, it should be understood that 64 MHz and 128 MHz are merely approximate values for the MRI frequencies.) Other components of the MRI, such as its sensing and imaging components are not shown. MRI systems and imaging techniques are well known and will not be described in detail herein. For exemplary MRI systems see, for example, U.S. Pat. No. 5,063,348 to Kuhara et al., entitled “Magnetic Resonance Imaging System” and U.S. Pat. No. 4,746,864 to Satoh, entitled “Magnetic Resonance Imaging System.” Note that the fields shown in FIG. 1 are stylized representations of MRI fields intended merely to illustrate the presence of the fields. Actual MRI fields generally have far more complex patterns.

### Lead Overview

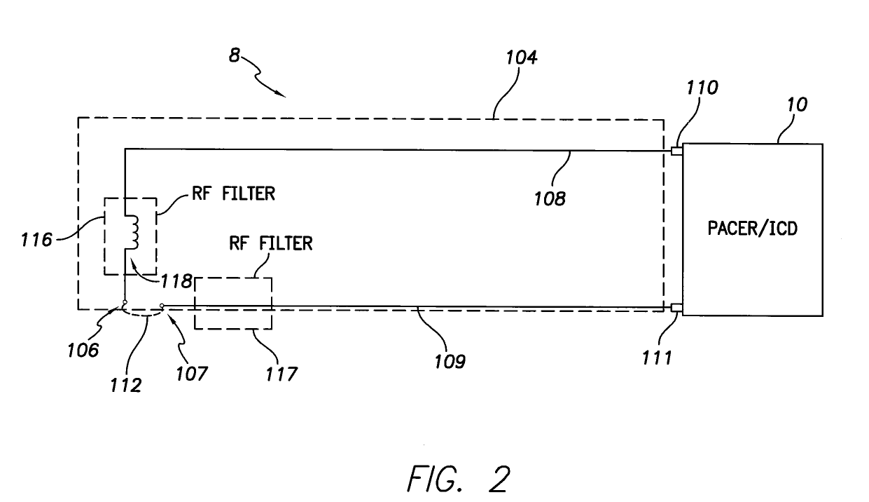

FIG. 2 illustrates an implantable system 8 having a pacer/ICD or other implantable medical device 10 with a bipolar coaxial lead 104. The bipolar lead includes a tip electrode 106 electrically connected to the pacer/ICD via a tip conductor 108 coupled to a tip connector or terminal 110 of the pacer/ICD. The bipolar lead also includes a ring electrode 107 electrically connected to the pacer/ICD via a ring conductor 109 coupled to a ring connector or terminal 111 of the pacer/ICD. Depending upon the particular implementation, during pacing/sensing, the tip electrode may be more negative than the ring, or vice versa. A conducting path 112 between tip electrode 106 and ring electrode 107 is provided through patient tissue (typically cardiac tissue.) An RF inductive filtering element 116 is connected along conductor 108 at a distal portion thereof near tip electrode 106. The RF filter includes, in this example, a single inductor 118. In other examples, more or fewer components may be used, including, e.g., filters including LCR circuits. Additionally or alternatively, one or more RF filters 117 may be connected along ring conductor 119. The various RF filtering components can be lumped or have a distributed structure.

With the lead arrangement of FIG. 2, during an MRI, one or more current loops might be induced within the lead (and within any circuit components within the pacer/ICD that electrically connect terminals 110 and 111). The RF filter is configured to block frequencies associated with such current loops to decrease the magnitude thereof. Without the RF filter, strong current loops might pass through patient tissue between the tip and ring electrodes before returning to the pacer/ICD, causing considerable resistive heating at the electrodes and in the intervening tissue. As explained above, such heating can damage patient tissue and interfere with pacing and sensing. In addition, as noted, the current loops can cause MRI-induced pacing.

With RF filters, however, any such current loops are greatly diminished, thereby reducing a significant source of tip or ring heating as well as preventing or limiting MRI-induced pacing. Although not shown, RF shielding and/or MRI responsive switches can also be provided to further reduce lead heating. Note that different types of RF filters may be provided within atrial leads as compared to ventricular leads, with the filters of ventricular leads being generally more robust than the RF filters of the atrial leads since, typically, larger currents are induced in ventricular leads than in atrial leads during an MRI. In the following sections, techniques will be described for selecting particular inductive components for use in the RF filter to achieve effective reduction in lead temperatures during an MRI within the patient or in the presence of other sources of strong RF fields.

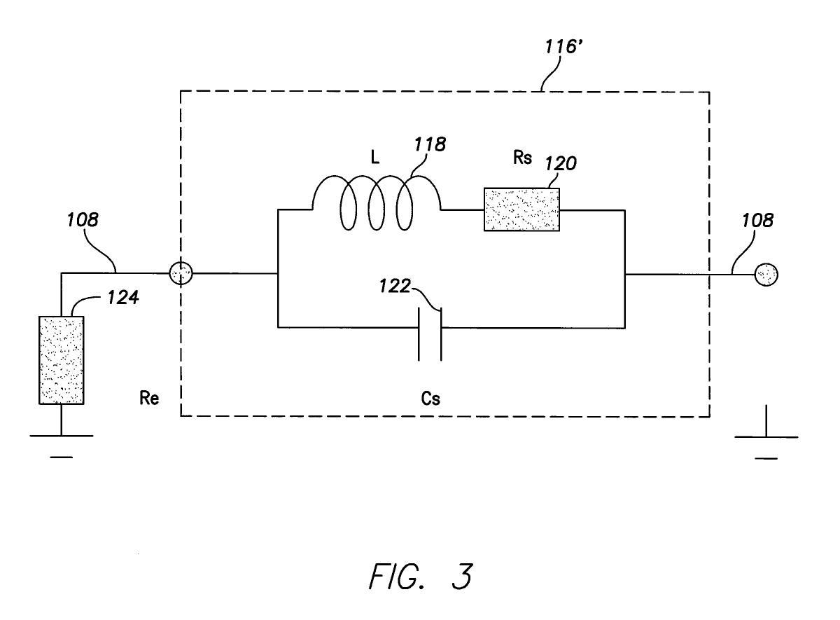

In this regard, although RF filter 116 includes only an inductor, parasitic capacitance (Cₛ) and parasitic resistances (Rₛ) are nevertheless present. FIG. 3 illustrates an equivalent circuit 116′ for RF filter 116, wherein the parasitic capacitance and parasitic resistance are schematically shown. The parasitic resistance 120 is in series with inductor 118 along conductor 108. The parasitic capacitance 122 is in parallel with the inductance/resistance along the conductor. The resistance of the current path (Re) is schematically illustrated by way of resistor block 124.

Accordingly, the parasitic capacitance 122 tends to affect the target impedance that can be achieved by the inductor at the RF signal frequencies, and hence the parasitic capacitance should also be taken into account when selecting the particular inductor to be used, whether designing an RF filter intended to operate effectively at both 64 MHz and 128 MHz or at just one of these MRI frequencies. Note also that the inductance (L) and parasitic capacitance (Cs) shown in FIG. 2 have a resonant frequency called the self-resonant frequency (SRF) as with separate L and C resonators. Inductors can be designed to have a desired SRF at specific frequencies such as at 64 MHz or 128 MHz or at a specific frequency there-between. Techniques for addressing these issues will be described in the next sections.

Note that the parasitic capacitance and the parasitic resistance of an inductor can be measured or determined from manufacturer specifications. Parasitic resistance and capacitance of inductors is discussed, e.g., in U.S. Pat. No. 6,395,637 to Park et al. of the Electronics and Telecommunications Research Institute, entitled “Method for Fabricating an Inductor of Low Parasitic Resistance and Capacitance.”

### Parasitic Capacitance-Based Inductor Selection for Use at One MRI Frequency

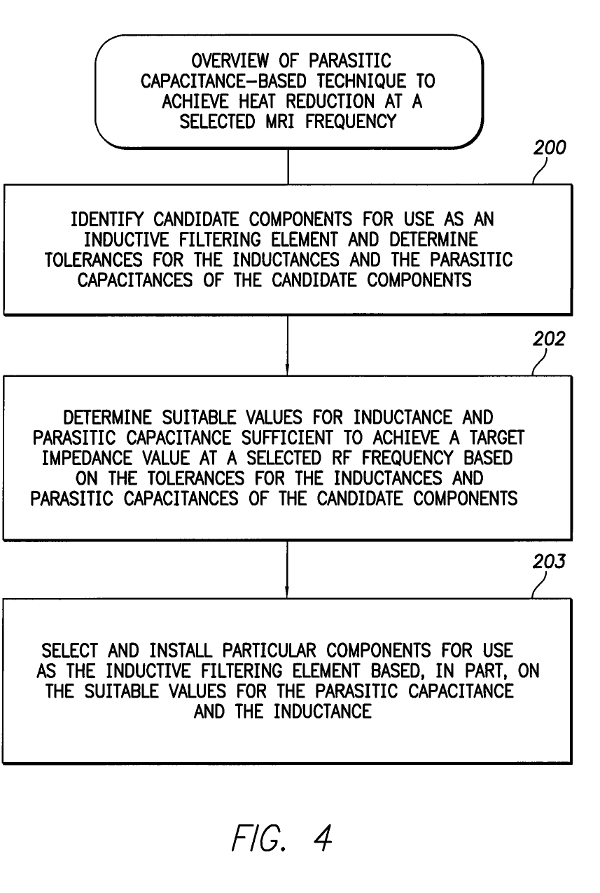

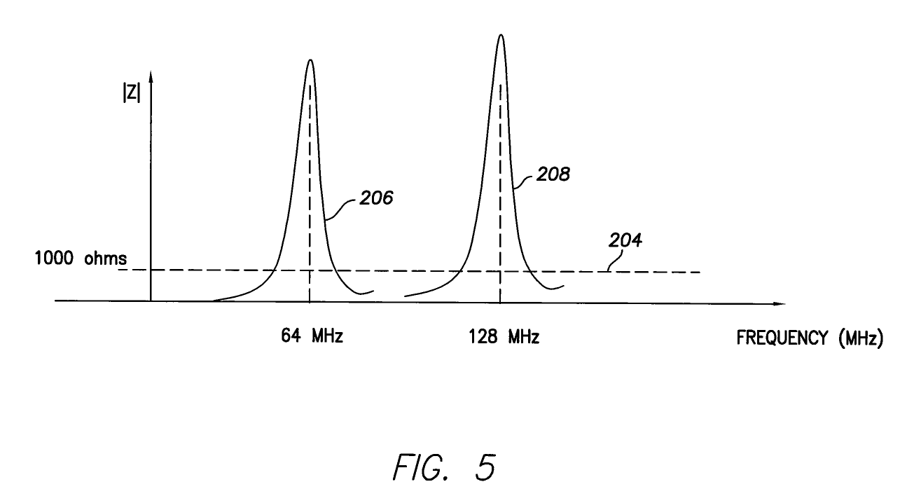

Turning now to FIGS. 4 and 5, an overview is provided of a technique for use by lead designers while designing or configuring a lead with an inductive RF filter wherein parasitic capacitance is taken into account to achieve adequate heat reduction at a selected MRI frequency, such as about 64 MHz or about 128 MHz. At step 200, candidate components are identified for use as the inductive RF filtering element and tolerances for the inductances and the parasitic capacitances of the candidate components are determined from manufacturer specifications. That is, candidate inductors for use as the RF filter of the lead are identified from among various components provided by manufactures (based, e.g., on size, weight, durability, etc.) and then the tolerances of the components are determined, e.g. with reference to manufacturer specifications or through independent testing.

At step 202, suitable values for inductance and parasitic capacitance are determined that are sufficient to achieve a target impedance value at a selected RF signal frequency, such as 64 MHz or 128 MHz, based on the tolerances for the inductances and parasitic capacitances of the candidate components. The target impedance value may be expressed as a targeted lower bound (Z₀) for the amplitude of the impedance. The target value may differ based on the characteristics of the RF fields to be filtered, such as its frequency. For a 64 MHz/1.5 T MRI or a 128 MHz/3.0 T MRI, an impedance value of 1000 ohms or greater is preferred. This value was ascertained via modeling/in-vitro tests to determine an impedance value sufficient to achieve significant heat reduction (within cardiac pacing/sensing leads wherein RF filtering components are connected in series with the tip and/or ring conductor and wherein the filtering components were not subject to significant variations due to device tolerance) and confirmed through experimentation. Higher target impedance values may also be used, such as 4000 ohms, if greater temperature reduction is needed. Typically, the higher the impedance, the better.

At step 203, particular components for use as the inductive filtering element are selected and installed based, in part, on the suitable values for the parasitic capacitance and the inductance determined at step 202. That is, at step 203, a particular model of inductor provided by a manufacturer is selected for use as the RF filter, wherein the selected inductor has an inductance and a parasitic capacitance sufficient to achieve the target impedance value at the selected RF signal frequency despite the variations in tolerance. The inductor is installed in leads and then implanted in patients for use with implantable medical devices (following suitable testing and approval procedures.) Hence, rather than merely selecting an inductor that, in theory, provides an SRF corresponding to the RF signals to be filtered, the technique of FIG. 4 operates to first determine suitable inductive and parasitic capacitive values while accounting for device tolerance, as well as the target impedance and the particular frequency to be filtered.

In one example, as shown in FIG. 5, the target impedance is set to 1000 ohms, as indicated by threshold 204. The goal is to determine L and Cs at step 202 so as to achieve at least that target impedance value at either 64 MHz or 128 MHz (but not necessarily both) despite variations in L or Cs due to device tolerance. In this regard, the SRF of an inductor provides an impedance curve centered at the SRF. For heat reduction at 64 MHz, the L and Cs values should be determined so as to center the curve at about 64 MHz, as shown by way of curve 206 to achieve the target impedance at that frequency. Alternatively, to achieve heat reduction at 128 MHz, the L and Cs values are determined so as to center the curve at 128 MHz, as shown by way of curve 208. If device tolerance were not a concern, it would be a simple matter of selecting an inductor having stated inductance and parasitic capacitance values so as to provide an SRF at or near 64 MHz sufficient to achieve 1000 ohms at that frequency (or to provide an SRF at or near 128 MHz sufficient to achieve 1000 ohms at that frequency). However, as already discussed, variations in actual L and Cs values due to device tolerance can reduce the impedance actually achieved at this frequencies. Hence, device tolerance should be taken into account at step 202. Parasitic resistance (Rs) can also be taken into account.

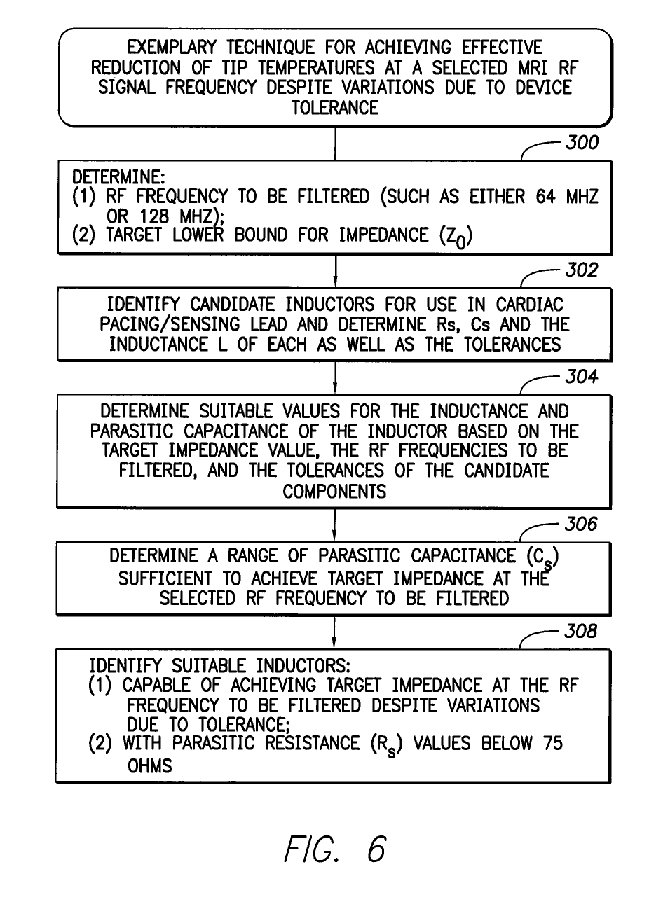

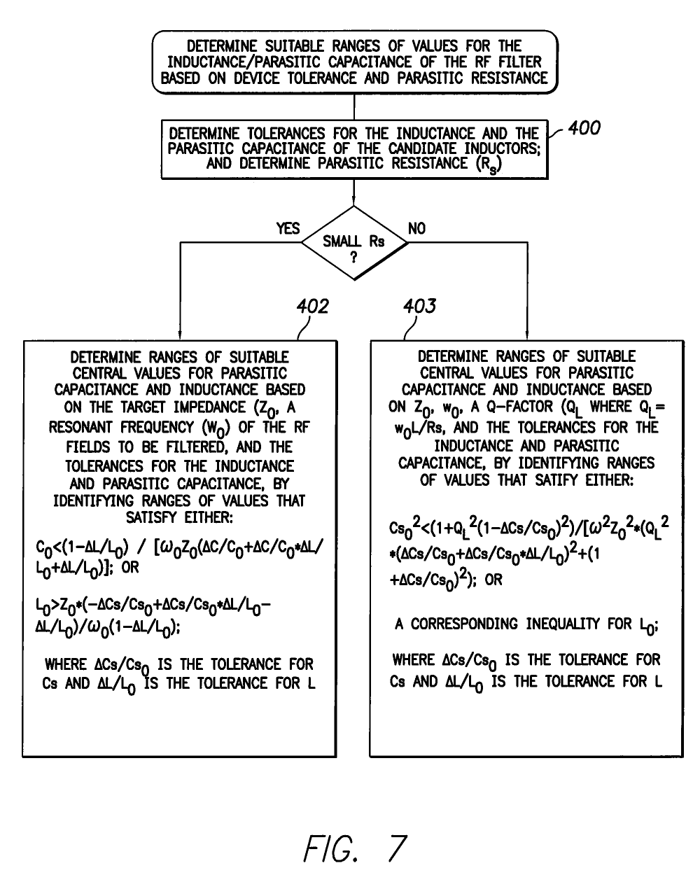

FIGS. 6 and 7 summarize component selection techniques for use by lead designers wherein self-resonant inductors are designed to have a single SRF at a specific RF and where parasitic resistance, parasitic capacitance, and device tolerances are all taken into account. Referring first to FIG. 6, at step 300, the following information is input or determined: (1) the RF signal frequency to be filtered (such as either 64 MHz or 128 MHz); and (2) the target lower bound for the impedance (Z₀), such as 2000 ohms. At step 302, candidate inductors are identified from among manufacturer-provided inductors or through customer-designed and the parasitic resistances (Rs), parasitic capacitances (Cs), and inductances (L) are determined for each, as well as the tolerances for both the inductance and the parasitic capacitance. The tolerances for Cs and L can be expressed as ΔCs/Cs₀ and ΔL/L₀, respectively, where Cs₀ represents a central value for the parasitic capacitance of the inductor and Lo represents a central value for the inductance of the inductor where the central values give the resonant frequency as the working frequency as ω²L₀Cs₀=1.

At step 304, suitable values for the inductance and parasitic capacitance of the inductor are determined based on the target impedance value (Z₀), the RF signal frequencies to be filtered, and the tolerances of the candidate components. Exemplary techniques for determining a suitable range of values for the inductance based on device tolerance will be described below. The range of values for inductance implies a corresponding range of values for the parasitic capacitance based on L₀=1/(ω₀²*Cs₀) where ω₀ is a resonant frequency (ω₀) of the RF fields to be filtered and, as noted, Cs₀ is the central value for the parasitic capacitance of the inductor. At step 306, a range of parasitic capacitance (Cₛ) is determined that is sufficient to achieve the target impedance at the selected frequency to be filtered.

Hence, following steps 300-306, suitable center inductance values have been identified that are sufficient to achieve the target impedance despite variations in actual inductance due to device tolerance. Moreover, a range of parasitic capacitance values has been identified that also satisfies the tolerance considerations. At step 308, suitable inductors are then identified: that are (1) capable of achieving the target impedance at the selected RF signal frequency to be filtered despite variations due to tolerance, and with (2) parasitic resistance (Rₛ) values below 75 ohms. That is, particular inductors are selected from among the candidate inductors that have inductance, parasitic resistance, parasitic capacitance, and tolerance values that collectively satisfy the various constraints already described. Assuming that at least some of the candidate inductors offer high inductance with good tolerance (i.e. with small values for ΔL/L₀), it is usually feasible to identify at least some inductors that meet the various constraints (at least at either about 64 MHz or at about 128 MHz.)

Turning now to FIG. 7, techniques for identifying a range of suitable values for inductance and parasitic capacitance based on device tolerance will be described for small Rs, i.e. Rs<<ωL/1000. The technique of FIG. 7 operates to determine a range of suitable values for the inductance and parasitic capacitance of the RF filtering SRF inductor based on a targeted lower bound for impedance, the parasitic resistance, and device tolerances for inductance and parasitic capacitance. Once a range of suitable inductance values has been determined, a corresponding value of suitable parasitic capacitance can be calculated. Alternatively, once a range of suitable parasitic capacitance values has been determined, a corresponding value of suitable inductance values can be calculated.

At step 400, the parasitic resistance (Rs) and device tolerances for inductance and parasitic capacitance of various candidate inductors are determined, either with reference to manufacturer specifications or via testing. The tolerance for inductance is generally represented herein as ΔL/L₀ (where L₀ is a center value for the inductance); whereas the tolerance for parasitic capacitance is generally represented herein as ΔCs/Cs₀ (where Cs₀ is a center value for the parasitic capacitance.)

If Rs is small (i.e. Q_{L}>1000, where Q_{L}=ω₀L/Rs), then, at step 402, a range of suitable central values (L₀) is determined for the inductance of the RF filter based on the targeted lower bound of impedance (Z₀), the resonant frequency (ω₀) of the RF fields to be filtered, and the tolerances for the parasitic capacitances and inductances of the inductor. That is, for each candidate inductor, a range of values are identified for L₀ that satisfy the condition:

L₀>Z₀*(−ΔCs/Cs₀+ΔCs/Cs₀*ΔL/L₀−ΔL/L₀)/ω₀(1−ΔL/L₀). (2)

Then, a corresponding range of values for Cs₀ can be determined based on L₀=1/(ω₀²*Cs₀). Alternatively, at step 402, a range of suitable parasitic capacitance values are first determined based on:

C₀<(1−ΔL/L₀)/[ω₀Z₀(ΔC/C₀+ΔC/C₀*ΔL/L₀+ΔL/L₀)] (3)

then the corresponding inductance values are calculated using L₀=1/(ω₀²*Cs₀). Although described with respect to self-resonant inductors with parasitic capacitance, similar techniques can be applied for use with LC circuits. See, U.S. patent application Ser. No. 11/955,268, filed Dec. 12, 2007, of Min, cited above.

Hence, using the criteria of either Equations (2) or (3), the selection of an inductor within the ranges of suitable pair of L₀ and Cs₀ would provide an amplitude of impedance greater than the target value (e.g. greater than the target lower amplitude bound of Z0, at least 1000 ohms and preferably 4000 ohms) with given tolerance (assuming small Rs). It should be understood that the tolerances of different candidate components will typically differ. Some may be ±5%, other ±10%, etc. The tolerances for parasitic capacitance need not be the same as that of inductance. Accordingly, given a collection of candidate inductors having differing tolerances for inductance and parasitic capacitance, Equations (2) or (3) may need to be tested for each pair of candidate inductors using the appropriate tolerance values. Also, due to practical size restrictions for the inductors and capacitors to be used within a lead, it is important to select components while taking into account mechanical considerations and other practical considerations.

Now considering circumstances where Rs is not small. Derived from equation (1) with Rs, |Z| with Rs can be expressed by L₀±ΔL and Cs₀±ΔCs as follows:

|Z|²=(1+Q_{L}²(1±ΔCs/Cs₀)²)/[ω₀²c₀²*(Q_{L}²*(±ΔCs/Cs₀±ΔCs/Cs₀*ΔL/L₀±ΔL/L₀)²+(1±ΔCs/Cs₀)²)>|Z₀|².

Using the equation, the minimum of |Z| is found by investigating the combinations of signs as follows:

Minimum |Z|²=(1+Q_{L}²(1−ΔCs/Cs₀)²)/[ω₀²c₀²*(Q_{L}²*(ΔCs/Cs₀+ΔCs/Cs₀*ΔL/L₀+ΔL/L₀ )²+(1+ΔCs/Cs₀)²)>|Z₀|²

Accordingly, at step 403, the following inequality (4) is evaluated to determine a range of suitable values for Cs₀ based on a selected value for L₀:

Cs₀²<(1+Q_{L}²(1−ΔCs/Cs₀)²)/[ω₀²Z₀²*(Q_{L}²*(ΔCs/Cs₀+ΔCs/Cs₀*ΔL/L₀+L/L₀)²+(1+ΔCs/Cs₀)²) (4)

where ω₀=1/sqrt(L₀Cs₀), Q_{L}=ω₀L/Rs and tolerance of ΔCs/Cs₀ and ΔL/L₀. Alternatively, a corresponding inequality for L₀² is evaluated for to determine a range of suitable values for L₀ based on a selected value for Cs₀. In either case, with manufacture-provided tolerance values, the inequalities guide inductor designs with L, Cs and Rs to satisfy the desired |Z₀| at a single selected MRI frequency. Again, although described with respect to self-resonant inductors with parasitic capacitance, similar techniques can be applied for use with LC circuits.

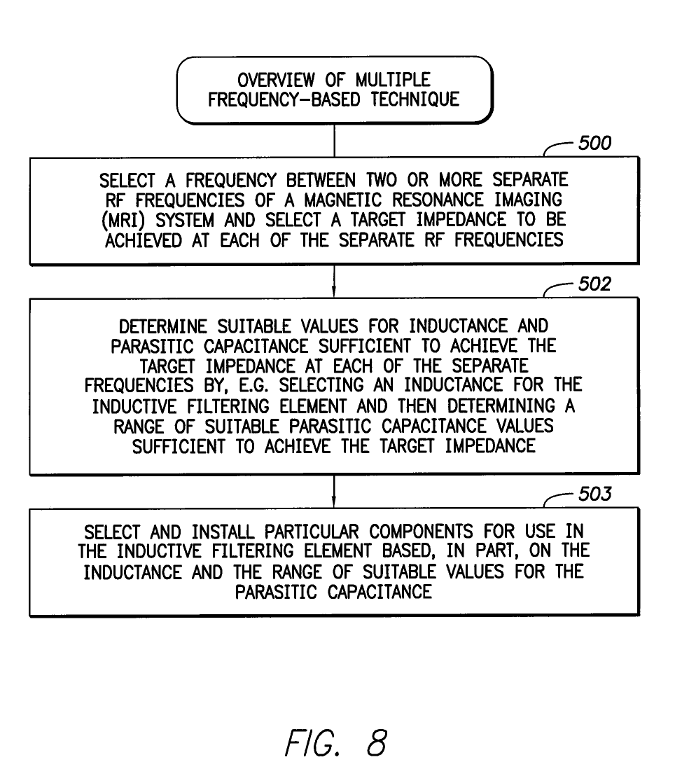

Turning now to FIGS. 8-10, techniques for achieving a target Z₀ at two or more separate MRI frequencies will be described.

### Parasitic Capacitance-Based Inductor Selection at Multiple MRI Frequencies

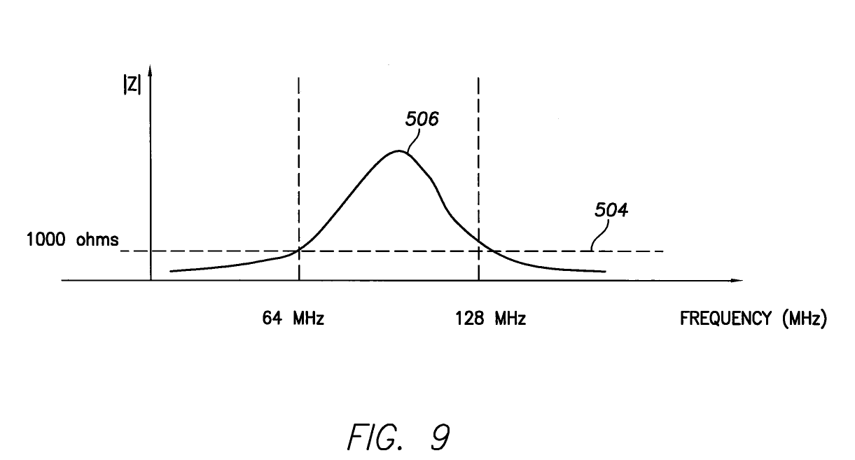

FIGS. 8 and 9 summarize a component selection technique for use by lead designers wherein parasitic capacitance is taken into account to achieve adequate heat reduction at a two or more selected MRI frequencies, such as at both 64 MHz and 128 MHz. Note that issues arising due to device tolerance are not specifically addressed within FIGS. 8 and 9 but are discussed below.

At step 500 of FIG. 8, a frequency is selected between two or more RF signal frequencies of an MRI system (such as midway between 64 Mhz and 128 Mhz) and a target impedance to be achieved at each of the separate RF signal frequencies is chosen (such as 1000 ohms.) At step 502, suitable values for inductance and parasitic capacitance are then determined that are sufficient to achieve the target impedance at each of the separate frequencies by, for example, selecting an inductance for the inductive filtering element and then determining a range of suitable parasitic capacitance values sufficient to achieve the target impedance. That is, at step 502, suitable values for inductance and parasitic capacitance are determined that are sufficient to achieve the target impedance value at both of the selected RF frequencies, such as at both 64 MHz and 128 MHz. At step 503, particular components for use as the inductive filtering element are selected and installed based, in part, on the suitable values for the parasitic capacitance and the inductance determined at step 502. That is, at step 503, a particular model of inductor provided by a manufacturer is selected for use as the RF filter, wherein the selected inductor has an inductance and a parasitic capacitance sufficient to achieve the target impedance value at both of the selected RF signal frequencies. The inductor is installed in leads and then implanted in patients for use with implantable medical devices (following suitable testing and approval procedures.) Hence, the technique of FIG. 8 operates to determine suitable inductive and parasitic capacitive values to achieve the desired impedance at both MRI RF signal frequencies to be filtered (or more generally at a set of three or more selected RF signal frequencies.)

In one example, as shown in FIG. 9, the target impedance is 1000 ohms, as indicated by threshold 504. The goal is to determine L and Cs at step 502 so as to achieve at least that target impedance value at both 64 MHz and 128 MHz. As already explained, the SRF of an inductor provides for an impedance curve centered at the SRF. For heat reduction at both 64 MHz and 128 MHz, the L and Cs values of the inductor are determined so as to center the curve somewhere between 64 MHz and 128 MHz, as shown by way of curve 506, while still achieving the target impedance at the frequencies of 64 MHz and 128 MHz. This is achieved by exploiting the techniques set forth below, which take L and Cs into account. Parasitic resistance (Rs) can also be taken into account. Also, rather than using an SRF inductor with Cs, separate inductors and capacitors can be employed, or LCR circuits, lumped or distributed inductors, etc.

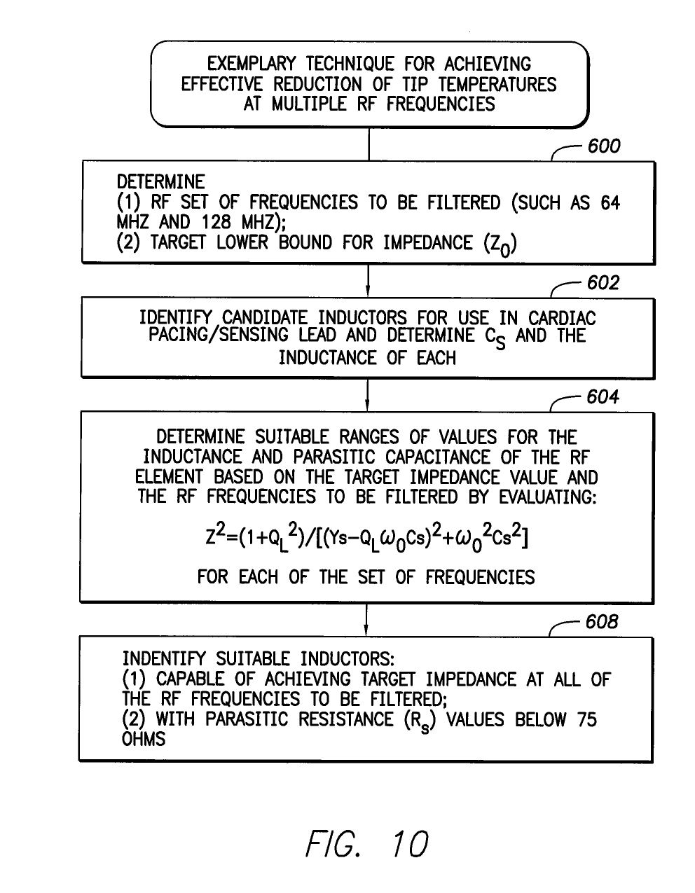

FIG. 10 summarizes a component selection technique for use by lead designers wherein self resonant inductors are designed to have a single SRF in between a pair of separate RF signal frequencies of an MRI. At step 600, the following information is input or determined: (1) the RF signal frequencies to be filtered (such as both 64 MHz and 128 MHz); and (2) the target lower bound for the impedance (Z₀), such as 2000 ohms. At step 602, candidate inductors are identified from among manufacturer-provided inductors or through customer-designed and parasitic capacitances (Cs) and inductances (L) are determined for each, as well, in some examples, the parasitic resistance Rs. Again, the tolerances for Cs and L are expressed as ΔCs/Cs₀ and ΔL/L₀.

At step 604, suitable ranges of values for the inductance and parasitic capacitance of the inductor are determined for each of the selected frequencies based on the target impedance value (Z₀) and the RF signal frequencies to be filtered by evaluating Equation (1) [listed above in the Summary and repeated here for convenience]:

Z²=(1+Q_{L}²)/[(YS−Q_{L}ω₀Cs)²+ω₀²Cs²]

where Ys=1/Rs and Q_{L}=ω₀L/Rs. Exemplary techniques for determining a suitable range of values for the inductance using this equation will be described below.

Hence, following steps 600-604, suitable ranges of values for inductance and parasitic capacitance have been identified that are sufficient to achieve the target impedance at two or more selected MRI frequencies. At step 608, suitable inductors are then identified: that are (1) capable of achieving the target impedance at all of the selected RF signal frequencies and with (2) parasitic resistance (Rₛ) values below 75 ohms. [For example, if f=64 MHz and ω=2*pi*64 MHz and L=3 uH, then Rs<<120 ohms, so that Rs<75 ohms.] Rs<50 ohms is preferred.

That is, particular inductors are selected from among the candidate inductors that have inductance, parasitic resistance, parasitic capacitance values that collectively satisfy the various constraints already described. Assuming that at least some of the candidate inductors offer high inductance with good tolerance (i.e. with small values for ΔL/L₀), it is usually feasible to identify at least some inductors that meet the various constraints (to achieve the target impedance at both 64 MHz and 128 MHz.)

Referring again to Equation (1), a set of inequalities are obtained (at step 604) by the conditions at |Z|>Z₀ at RF=64 MHz and |Z|>Z₀ at 128 MHz. Then parasitic capacitance values are determined that will yield an impedance greater than the target lower bound of impedance (Z₀). One exemplary technique to determine the parameter selection is to use a simplified formula with Rs=0 [Equation (5), below] and then set |Z|>|Z₀| with |Z₀| higher than the desired value. That is, set Z₀″ higher than Z₀ i.e. |Z|>|Z₀″| and verify L and Cs values>|Z₀| by Equation (1) with Rs.

|Z|=ωL/abs(1−ω²LCs) (5)

Given the absolute value within equation (5), each combination of values for ω₀ and L leads to a separate range of acceptable values for Cₛ sufficient to achieve a Z value exceeding Z₀.

Table I sets forth the ranges of acceptable values of Cₛ for various values of L, Cs and ω₀ with the target impedance Z₀ when Z₀′ is set to 1050 ohms for equation (5) and verified |Z|>|Z₀|=1000 ohms with Rs=0 ohms to 75 ohms. |Z|>|Z₀|=1000 ohms at 64 MHz and 128 MHz and for the selected L values listed in the table, Cs needs to satisfy all the conditions listed in Table 1 for selected L. In the table, for each target frequency, a first group of Cₛ values represent the range derived when Equation (5) is solved for Cₛ with ωL₀Cs₀>1; whereas the second group of Cₛ values represent the range derived when Equation (1) is solved for Cₛ with ω₀L₀Cs₀<1.

TABLE I

| RF freq. | | | | | | | |
|---|---|---|---|---|---|---|---|
| (MHz) | ω₀ | Z₀/Zₒ(Rs) | Cₛ (pF)< | L (nH) | ωL₀C₀ > 1 | Cₛ(pf)> | L₀/Cₛ ratios |
| 65.3 | 410.3 | 1050/1000 | 5.5 | 2000 | 1.76 | 3.12 | 363.58 |
| | | 1050/1000 | 5.85 | 1800 | 1.69 | 3.47 | 307.82 |
| | | 1050/1000 | 6.5 | 1500 | 1.57 | 4.16 | 229.32 |
| RF freq. | | | | | | | |
| (MHz) | ω₀ | Z₀/Zₒ(Rs) | Cₛ (pF)> | L (nH) | ωL₀C₀ < 1 | Cₛ(pf)< | L₀/Cₛ ratios |
| 65.3 | 410.3 | 1050/1000 | 0.74 | 2000 | 0.24 | 3.12 | 2696.4 |
| | | 1050/1000 | 1.09 | 1800 | 0.31 | 3.47 | 1653.60 |
| | | 1050/1000 | 1.78 | 1500 | 0.43 | 4.16 | 841.68 |
| RF freq. | | | | | | | |
| (MHz) | ω₀ | Z₀/Zₒ(Rs) | Cₛ (pF)< | L (nH) | ωL₀C₀ > 1 | Cₛ(pf)> | L₀/Cₛ ratios |
| 128 | 804.25 | 1050/1000 | 1.96 | 2000 | 2.53 | 0.77 | 1021.87 |
| | | 1050/1000 | 2.04 | 1800 | 2.61 | 0.86 | 881.02 |
| | | 1050/1000 | 2.21 | 1500 | 2.15 | 1.03 | 677.24 |
| RF freq. | | | | | | | |
| (MHz) | ω₀ | Z₀/Zₒ(Rs) | Cₛ (pF)> | L (nH) | ωL₀C₀ < 1 | Cₛ(pf)< | L₀/Cₛ ratios |
| 128 | 804.25 | 1050/1000 | −0.41 | 2000 | −0.53 | 0.77 | −4864.17 |
| | | 1050/1000 | −0.33 | 1800 | −0.38 | 0.86 | −5533.70 |
| | | 1050/1000 | −0.15 | 1500 | −0.15 | 1.03 | −9772.1 |

For example, with L=2000 nH, the suitable range for Cₛ at 63.7 MHz is 0.74 pF<Cₛ<3.12 pF and 3.12 pF<Cₛ<5.5 pF (i.e. 0.74 pF<Cs <5.5 pF). That is, a 2000 nH inductor having a parasitic capacitance anywhere in that range will meet or exceed the lower bound impedance target of 1000 ohms at the RF signal frequency of 65.3 MHz. For a frequency of 128 MHz, however, with L =2000 nH, the range for Cs is 0.77 pF<Cs<1.96 pF and −0.41 pF<Cs<0.77 pF (i.e. −0.41 pF<Cs<1.96 pf) (with any negative capacitance values ignored). Hence, in order to meet the target impedance at both 65.3 MHz and 128 MHz, the acceptable range for Cs for a 2000 nH inductor is the overlap of the separate ranges, i.e., 0.77<Cs<1.96.

Table II summarizes the acceptable ranges of Cs for the various examples of Table I. As can be seen, for an inductor with L of 1000 nH, there is no overlap in the parasitic capacitance ranges. That is, the target impedance of 1000 ohms cannot be achieved at both 65.3 MHz and 128 MHz.

TABLE II

| L (nH) | Parasitic Cₛ (pF) | Parasitic Rₛ (ohms) | Comments |
|---|---|---|---|
| 2000 | 0.77 < Cₛ < 1.96 | 0 <= Rₛ < 75 ohms | Suitable for both |
| | | | 65.3 MHz and |
| | | | 128 MHz. |
| 1800 | 1.09 < Cₛ < 2.04 | 0 <= Rₛ < 75 ohms | Suitable for both |
| | | | 65.3 MHz and |
| | | | 128 MHz. |
| 1500 | 1.78 < Cₛ < 2.2 | 0 <= Rₛ < 75 ohms | Suitable for both |
| | | | 65.3 MHz and |
| | | | 128 MHz. |
| 1000 | 1.5T: 3.5 < Cₛ < 8.3 | N/A | No a common range |
| | 3.0T: 0.3 < Cₛ < 2.8 | | for both 65.3 MHz |
| | | | and 128 MHz. |

As noted, at step 608, suitable inductors are identified from among the candidate inductors that have parasitic capacitance (Cs) values within the range of suitable values determined from the inequalities and which have sufficient inductance to achieve the target impedance. For example, a 2000 nH inductor provided by a manufacturer having a Cs value in the range of 0.77<Cs<1.96 would be effective for use in the RF filter (assuming it is otherwise suitable based on size, weight, durability, etc.) The RF element is then fabricated or built using the selected inductor and installed in a lead for eventual implant in a patient for use with an implantable medical device (following any necessary FDA or other government approval.) In order to achieve greater RF heating reduction, higher impedance Z may be used such as |Z|>2000 ohms to ensure temperature increase less than 3 degree in gel phantom at the non-clinical configurations during MRI scans.

In some implementations, it is also desirable to take into account the device tolerances of the inductor, in addition to the aforementioned parasitic capacitance and parasitic resistance constraints. This is accomplished by generating a set of inequalities based on tolerance, then identifying ranges of values for L and Cs that satisfy the inequalities. For example, given a central L₀ and a known ΔL, either Cs or component C can be identified through the set of inequalities derived from the equation in Equation (1). Then the common zone for Cs (ignoring tolerance in Cs) is the solution for L₀±ΔL and Cs to meet |Z| criteria. That is:

| | | |
|---|---|---|
| L₀ | leads to | a₀ < Cs < b₀ |
| L₀ + ΔL | leads to | a₁ < Cs < b₁ |
| L₀ − ΔL | leads to | a₂ < Cs < b₂ |

The solution is A<Cs<B where A=max (a₀, a₁, a₂) and B=min (b₀, b₁, b₂).

When taking Cs tolerance into account (Cs±ΔCs), the solution to meet |Z| criteria is:

A<(Cs−ΔCs) AND (Cs+ΔCs)<B

Although described primarily with respect to an inductor having an inductance and a parasitic resistance, the techniques described herein are applicable, where appropriate, to inductive-capacitive elements having both an inductor and a capacitor, as well as to LCR circuits, lumped inductors, etc.

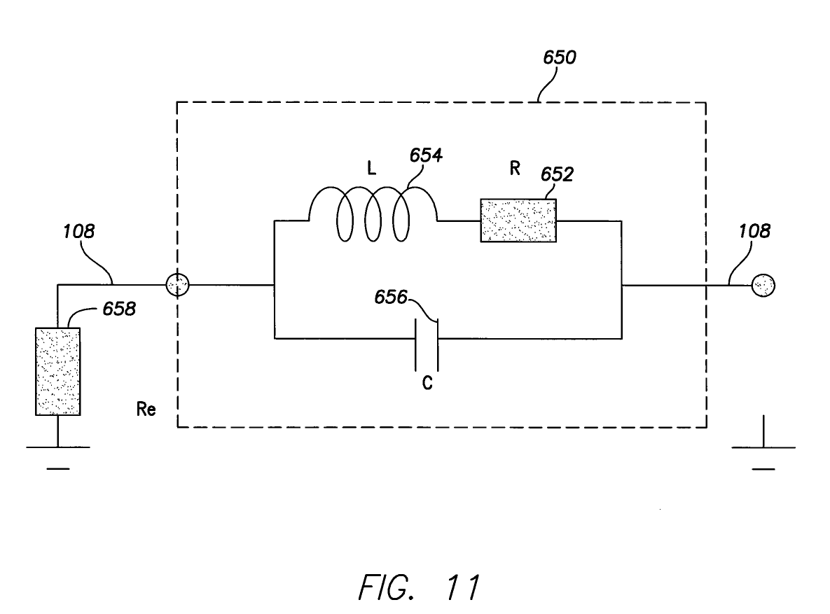

FIG. 11 illustrates an LCR 650 for use as the RF filter of FIG. 2. A resistor 652 is in series with an inductor 654 along conductor 108. A capacitor 656 is in parallel with the inductance/resistance along the conductor. The resistance of the current path (Re) is schematically illustrated by way of resistor block 658. In still other implementations, the RF filter includes an inductor and a capacitor, but the resistance is parasitic. Lumped inductors can also be employed, where appropriate.

RF filters designed using the techniques described herein can be exploited for use with a wide variety of leads of implantable medical systems. For the sake of completeness, a detailed description of an exemplary pacer/ICD and lead system will now be provided.

### Exemplary Pacer/ICD/Lead System

FIG. 12 provides a simplified diagram of the pacer/ICD of FIG. 1, which is a dual-chamber stimulation device capable of treating both fast and slow arrhythmias with stimulation therapy, including cardioversion, defibrillation, and pacing stimulation. To provide atrial chamber pacing stimulation and sensing, pacer/ICD 10 is shown in electrical communication with a heart 712 by way of a left atrial lead 720 having an atrial tip electrode 722 and an atrial ring electrode 723 implanted in the atrial appendage. Pacer/ICD 10 is also in electrical communication with the heart by way of a right ventricular lead 730 having, in this embodiment, a ventricular tip electrode 732, a right ventricular ring electrode 734, a right ventricular (RV) coil electrode 736. Typically, the right ventricular lead 730 is transvenously inserted into the heart so as to place the RV coil electrode 736 in the right ventricular apex. Accordingly, the right ventricular lead is capable of receiving cardiac signals, and delivering stimulation in the form of pacing and shock therapy to the right ventricle. An RF filtering element 716, designed as described above, is positioned within lead 730 near tip electrode 732 for use in attenuating high frequency signals so as to reduce lead heating. In the figure, the RF filtering element is shown in phantom lines as it is internal to the lead.

To sense left atrial and ventricular cardiac signals and to provide left chamber pacing therapy, pacer/ICD 10 is coupled to a “coronary sinus” lead 724 designed for placement in the “coronary sinus region” via the coronary sinus os for positioning a distal electrode adjacent to the left ventricle and/or additional electrode(s) adjacent to the left atrium. As used herein, the phrase “coronary sinus region” refers to the vasculature of the left ventricle, including any portion of the coronary sinus, great cardiac vein, left marginal vein, left posterior ventricular vein, middle cardiac vein, and/or small cardiac vein or any other cardiac vein accessible by the coronary sinus. Accordingly, an exemplary coronary sinus lead 724 is designed to receive atrial and ventricular cardiac signals and to deliver left ventricular pacing therapy using at least a left ventricular tip electrode 726 and a left ventricular ring electrode 729 and to deliver left atrial pacing therapy using at least a left atrial ring electrode 727, and shocking therapy using at least an SVC coil electrode 728. An RF filtering element 717, designed as described above, is positioned within lead 724 near tip electrode 726 for use in attenuating high frequency signals so as to reduce lead heating. In the figure, the RF filtering element is shown in phantom lines as it is internal to the lead.

With this configuration, biventricular pacing can be performed. Although only three leads are shown in FIG. 12, it should also be understood that additional stimulation leads (with one or more pacing, sensing and/or shocking electrodes) may be used in order to efficiently and effectively provide pacing stimulation to the left side of the heart or atrial cardioversion and/or defibrillation.

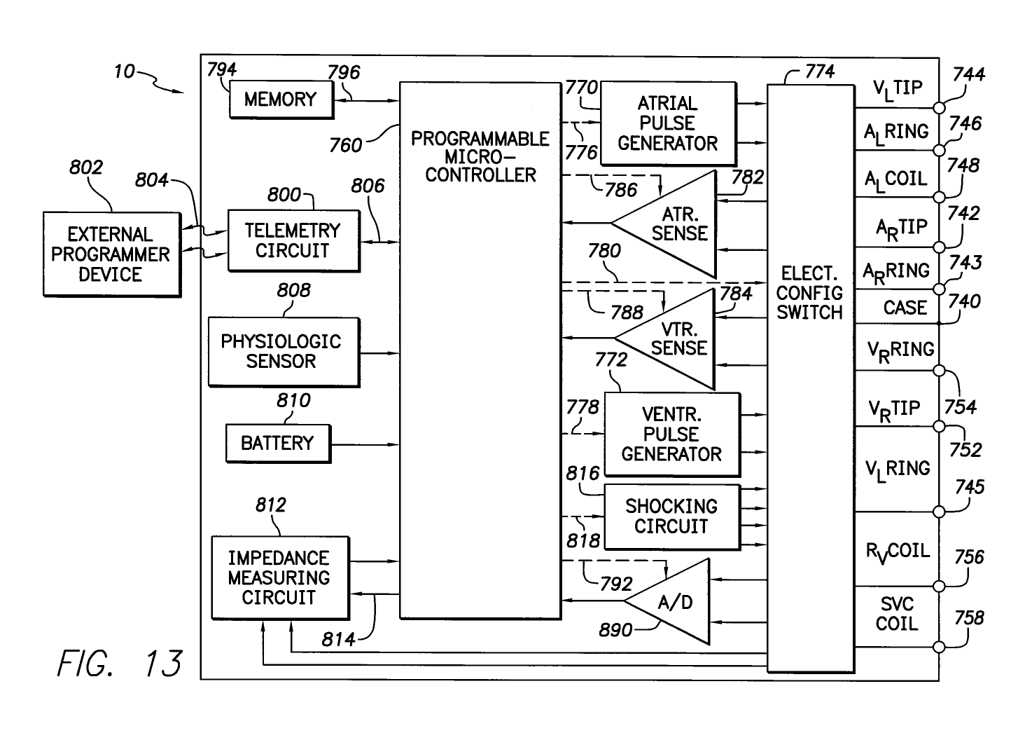

A simplified block diagram of internal components of pacer/ICD 10 is shown in FIG. 13. While a particular pacer/ICD is shown, this is for illustration purposes only, and one of skill in the art could readily duplicate, eliminate or disable the appropriate circuitry in any desired combination to provide a device capable of treating the appropriate chamber(s) with cardioversion, defibrillation and pacing stimulation as well as providing for the aforementioned apnea detection and therapy.

The housing 740 for pacer/ICD 10, shown schematically in FIG. 13, is often referred to as the “can”, “case” or “case electrode” and may be programmably selected to act as the return electrode for all “unipolar” modes. The housing 740 may further be used as a return electrode alone or in combination with one or more of the coil electrodes, 728, 736 and 738, for shocking purposes. The housing 740 further includes a connector (not shown) having a plurality of terminals, 742, 743, 744, 745, 746, 748, 752, 754, 756 and 758 (shown schematically and, for convenience, the names of the electrodes to which they are connected are shown next to the terminals). As such, to achieve right atrial sensing and pacing, the connector includes at least a right atrial tip terminal (A_{R} TIP) 742 adapted for connection to the atrial tip electrode 722 and a right atrial ring (A_{R} RING) electrode 743 adapted for connection to right atrial ring electrode 723. To achieve left chamber sensing, pacing and shocking, the connector includes at least a left ventricular tip terminal (V_{L} TIP) 744, a left ventricular ring terminal (V_{L} RING) 745, a left atrial ring terminal (A_{L} RING) 746, and a left atrial shocking terminal (A_{L} COIL) 748, which are adapted for connection to the left ventricular ring electrode 726, the left atrial tip electrode 727, and the left atrial coil electrode 728, respectively. To support right chamber sensing, pacing and shocking, the connector further includes a right ventricular tip terminal (V_{R} TIP) 752, a right ventricular ring terminal (V_{R} RING) 754, a right ventricular shocking terminal (R_{V} COIL) 756, and an SVC shocking terminal (SVC COIL) 758, which are adapted for connection to the right ventricular tip electrode 732, right ventricular ring electrode 734, the RV coil electrode 736, and the SVC coil electrode 738, respectively.

At the core of pacer/ICD 10 is a programmable microcontroller 760, which controls the various modes of stimulation therapy. As is well known in the art, the microcontroller 760 (also referred to herein as a control unit) typically includes a microprocessor, or equivalent control circuitry, designed specifically for controlling the delivery of stimulation therapy and may further include RAM or ROM memory, logic and timing circuitry, state machine circuitry, and I/O circuitry. Typically, the microcontroller 760 includes the ability to process or monitor input signals (data) as controlled by a program code stored in a designated block of memory. The details of the design and operation of the microcontroller 760 are not critical to the invention. Rather, any suitable microcontroller 760 may be used that carries out the functions described herein. The use of microprocessor-based control circuits for performing timing and data analysis functions are well known in the art.

As shown in FIG. 13, an atrial pulse generator 770 and a ventricular pulse generator 772 generate pacing stimulation pulses for delivery by the right atrial lead 720, the right ventricular lead 730, and/or the coronary sinus lead 724 via an electrode configuration switch 774. It is understood that in order to provide stimulation therapy in each of the four chambers of the heart, the atrial and ventricular pulse generators, 770 and 772, may include dedicated, independent pulse generators, multiplexed pulse generators or shared pulse generators. The pulse generators, 770 and 772, are controlled by the microcontroller 760 via appropriate control signals, 776 and 778, respectively, to trigger or inhibit the stimulation pulses.

The microcontroller 760 further includes timing control circuitry (not separately shown) used to control the timing of such stimulation pulses (e.g., pacing rate, atrio-ventricular (AV) delay, atrial interconduction (A-A) delay, or ventricular interconduction (V-V) delay, etc.) as well as to keep track of the timing of refractory periods, blanking intervals, noise detection windows, evoked response windows, alert intervals, marker channel timing, etc., which is well known in the art. Switch 774 includes a plurality of switches for connecting the desired electrodes to the appropriate I/O circuits, thereby providing complete electrode programmability. Accordingly, the switch 774, in response to a control signal 780 from the microcontroller 760, determines the polarity of the stimulation pulses (e.g., unipolar, bipolar, combipolar, etc.) by selectively closing the appropriate combination of switches (not shown) as is known in the art.

Atrial sensing circuits 782 and ventricular sensing circuits 784 may also be selectively coupled to the right atrial lead 720, coronary sinus lead 724, and the right ventricular lead 730, through the switch 774 for detecting the presence of cardiac activity in each of the four chambers of the heart. Accordingly, the atrial (ATR. SENSE) and ventricular (VTR. SENSE) sensing circuits, 782 and 784, may include dedicated sense amplifiers, multiplexed amplifiers or shared amplifiers. The switch 774 determines the “sensing polarity” of the cardiac signal by selectively closing the appropriate switches, as is also known in the art. In this way, the clinician may program the sensing polarity independent of the stimulation polarity. Each sensing circuit, 782 and 784, preferably employs one or more low power, precision amplifiers with programmable gain and/or automatic gain control and/or automatic sensitivity control, bandpass filtering, and a threshold detection circuit, as known in the art, to selectively sense the cardiac signal of interest. The automatic gain and/or sensitivity control enables pacer/ICD 10 to deal effectively with the difficult problem of sensing the low amplitude signal characteristics of atrial or ventricular fibrillation. The outputs of the atrial and ventricular sensing circuits, 782 and 784, are connected to the microcontroller 760 which, in turn, are able to trigger or inhibit the atrial and ventricular pulse generators, 770 and 772, respectively, in a demand fashion in response to the absence or presence of cardiac activity in the appropriate chambers of the heart.

For arrhythmia detection, pacer/ICD 10 utilizes the atrial and ventricular sensing circuits, 782 and 784, to sense cardiac signals to determine whether a rhythm is physiologic or pathologic. As used herein “sensing” is reserved for the noting of an electrical signal, and “detection” is the processing of these sensed signals and noting the presence of an arrhythmia. The timing intervals between sensed events (e.g., P-waves, R-waves, and depolarization signals associated with fibrillation which are sometimes referred to as “Fib-waves”) are then classified by the microcontroller 760 by comparing them to a predefined rate zone limit (i.e., bradycardia, normal, atrial tachycardia, atrial fibrillation, low rate VT, high rate VT, and fibrillation rate zones) and various other characteristics (e.g., sudden onset, stability, physiologic sensors, and morphology, etc.) in order to determine the type of remedial therapy that is needed (e.g., bradycardia pacing, antitachycardia pacing, cardioversion shocks or defibrillation shocks).

Cardiac signals are also applied to the inputs of an analog-to-digital (A/D) data acquisition system 790. The data acquisition system 790 is configured to acquire intracardiac electrogram signals, convert the raw analog data into a digital signal, and store the digital signals for later processing and/or telemetric transmission to an external device 802. The data acquisition system 790 is coupled to the right atrial lead 720, the coronary sinus lead 724, and the right ventricular lead 730 through the switch 774 to sample cardiac signals across any pair of desired electrodes. The microcontroller 760 is further coupled to a memory 794 by a suitable data/address bus 796, wherein the programmable operating parameters used by the microcontroller 760 are stored and modified, as required, in order to customize the operation of pacer/ICD 10 to suit the needs of a particular patient. Such operating parameters define, for example, pacing pulse amplitude or magnitude, pulse duration, electrode polarity, rate, sensitivity, automatic features, arrhythmia detection criteria, and the amplitude, waveshape and vector of each shocking pulse to be delivered to the patient's heart within each respective tier of therapy. Other pacing parameters include base rate, rest rate and circadian base rate.

Advantageously, the operating parameters of the implantable pacer/ICD 10 may be non-invasively programmed into the memory 794 through a telemetry circuit 800 in telemetric communication with an external device 802, such as a programmer, transtelephonic transceiver or a diagnostic system analyzer, or a bedside monitoring system. The telemetry circuit 800 is activated by the microcontroller by a control signal 806. The telemetry circuit 800 advantageously allows IEGMs and other electrophysiological signals and/or hemodynamic signals and status information relating to the operation of pacer/ICD 10 (as stored in the microcontroller 760 or memory 794) to be sent to the external programmer device 802 through an established communication link 804.

Pacer/ICD 10 further includes an accelerometer or other physiologic sensor 808, commonly referred to as a “rate-responsive” sensor because it is typically used to adjust pacing stimulation rate according to the exercise state of the patient. However, the physiological sensor 808 may further be used to detect changes in cardiac output, changes in the physiological condition of the heart, or diurnal changes in activity (e.g., detecting sleep and wake states) and to detect arousal from sleep. Accordingly, the microcontroller 760 responds by adjusting the various pacing parameters (such as rate, AV Delay, V-V Delay, etc.) at which the atrial and ventricular pulse generators, 770 and 772, generate stimulation pulses. While shown as being included within pacer/ICD 10, it is to be understood that the physiologic sensor 808 may also be external to pacer/ICD 10, yet still be implanted within or carried by the patient. A common type of rate responsive sensor is an activity sensor incorporating an accelerometer or a piezoelectric crystal, which is mounted within the housing 740 of pacer/ICD 10. Other types of physiologic sensors are also known, for example, sensors that sense the oxygen content of blood, respiration rate and/or minute ventilation, pH of blood, ventricular gradient, etc.

The pacer/ICD additionally includes a battery 810, which provides operating power to all of the circuits shown in FIG. 13. The battery 810 may vary depending on the capabilities of pacer/ICD 10. If the system only provides low voltage therapy, a lithium iodine or lithium copper fluoride cell may be utilized. For pacer/ICD 10, which employs shocking therapy, the battery 810 must be capable of operating at low current drains for long periods, and then be capable of providing high-current pulses (for capacitor charging) when the patient requires a shock pulse. The battery 810 must also have a predictable discharge characteristic so that elective replacement time can be detected. Accordingly, pacer/ICD 10 is preferably capable of high voltage therapy and appropriate batteries.

As further shown in FIG. 13, pacer/ICD 10 is shown as having an impedance measuring circuit 812 which is enabled by the microcontroller 760 via a control signal 814. Various uses for an impedance measuring circuit include, but are not limited to, lead impedance surveillance during the acute and chronic phases for proper lead positioning or dislodgement; detecting operable electrodes and automatically switching to an operable pair if dislodgement occurs; measuring respiration or minute ventilation; measuring thoracic impedance for determining shock thresholds; detecting when the device has been implanted; measuring respiration; and detecting the opening of heart valves, measuring lead resistance, etc. The impedance measuring circuit 120 is advantageously coupled to the switch 84 so that any desired electrode may be used.

In the case where pacer/ICD 10 is intended to operate as an implantable cardioverter/defibrillator (ICD) device, it detects the occurrence of an arrhythmia, and automatically applies an appropriate electrical shock therapy to the heart aimed at terminating the detected arrhythmia. To this end, the microcontroller 760 further controls a shocking circuit 816 by way of a control signal 818. The shocking circuit 816 generates shocking pulses of low (up to 0.5 joules), moderate (0.5-11 joules) or high energy (11 to at least 40 joules), as controlled by the microcontroller 760. Such shocking pulses are applied to the heart of the patient through at least two shocking electrodes, and as shown in this embodiment, selected from the left atrial coil electrode 728, the RV coil electrode 736, and/or the SVC coil electrode 738. The housing 740 may act as an active electrode in combination with the RV electrode 736, or as part of a split electrical vector using the SVC coil electrode 738 or the left atrial coil electrode 728 (i.e., using the RV electrode as a common electrode). Cardioversion shocks are generally considered to be of low to moderate energy level (so as to minimize pain felt by the patient), and/or synchronized with an R-wave and/or pertaining to the treatment of tachycardia. Defibrillation shocks are generally of moderate to high energy level (i.e., corresponding to thresholds in the range of 11-40 joules), delivered asynchronously (since R-waves may be too disorganized), and pertaining exclusively to the treatment of fibrillation. Accordingly, the microcontroller 760 is capable of controlling the synchronous or asynchronous delivery of the shocking pulses.

What have been described are systems and methods for use with pacing/sensing leads for use with a pacer/ICD. Principles of the invention may be exploiting using other implantable systems or in accordance with other techniques. Thus, while the invention has been described with reference to particular exemplary embodiments, modifications can be made thereto without departing from the scope of the invention.

What is claimed is:

1. A method for designing a lead for use with an implantable medical device, wherein the lead includes an inductive filtering element to reduce lead heating due to radio-frequency (RF) fields, the inductive filtering element having an inductance and a parasitic capacitance, the method comprising:
identifying candidate components for use as the inductive filtering element and determining tolerances for the inductances and the parasitic capacitances of the candidate components;
determining suitable values for inductance and parasitic capacitance sufficient to achieve a target impedance value at a selected frequency based on the tolerances for the inductances and parasitic capacitances of the candidate components; and
selecting and installing particular components for use as the inductive filtering element based, in part, on the suitable values for the parasitic capacitance and the inductance.

2. The method of claim 1 wherein the selected frequency is in the range of 63.7±0.345 MHz.

3. The method of claim 1 wherein the selected frequency is in the range of 127.6±3.6 MHz.

4. The method of claim 1 wherein the candidate components are inductors and wherein selecting and installing particular components for use as the inductive filtering element includes selecting a particular inductor from among a set of candidate inductors wherein the selected inductor has an inductance and a parasitic capacitance sufficient to achieve the target impedance value at the selected RF signal frequency despite variations due to tolerance.

5. The method of claim 1 wherein the target impedance value is a targeted lower bound (Z₀) for the amplitude of the impedance for the inductive filtering element at a resonant frequency determined based on the frequency to be filtered.

6. The method of claim 5 wherein the target impedance is at least 1000 ohms.

7. The method of claim 5 wherein determining values for the inductances and parasitic capacitances of the inductive filtering element includes determining ranges of suitable values for parasitic capacitance by identifying values satisfying the condition that:
Csₒ<(1 −ΔL/L₀)/[ω₀Z₀(ΔCs/Cs₀+ΔCs/Cs₀*ΔL/L₀+ΔL/L₀)]
where Z₀ represents a target lower bound for the impedance, Cs₀ represents a suitable central value for the parasitic capacitance Cs, L₀ represents a suitable central inductance value, ΔCs/Cs₀ represents the tolerance of the parasitic capacitance of the filtering element, ΔL/L₀ represents the tolerance of the inductance of the filtering element, and ω₀ represents the resonant frequency.

8. The method of claim 7 determining the range for inductance of the filtering element includes identifying particular combinations of L₀ and Cs₀ values that satisfy ω₀²=1/(Cs₀*L₀).

9. The method of claim 8 wherein selecting components for use in the filtering element based on the suitable values for the inductance and parasitic capacitance includes identifying particular components from among the candidate components having central inductance and parasitic capacitance values corresponding to the particular combinations of suitable L₀ and Cs₀ values.

10. The method of claim 5 wherein determining values for the inductance and parasitic capacitance of the filtering element includes determining ranges of suitable values for inductance by identifying values satisfying the condition that:
L₀>Z₀*(ΔCs/Cs₀+ΔCs/Cs₀*ΔL/L₀+ΔL/L₀)/ω₀(1−ΔL/L₀)
where Z₀ represents a target lower bound for the impedance, Cs₀ represents a suitable central value for the parasitic capacitance, L₀ represents a suitable central inductance value, ΔCs/Cs₀ represents the tolerance of the parasitic capacitance of the filtering element, ΔL/L₀ represents the tolerance of the inductance of the filtering element, and ω₀ represents the resonant frequency.

11. The method of claim 10 determining the range for parasitic capacitance of the filtering element further includes identifying particular combinations of paired L₀ and Cs₀ values that satisfy ω₀²=1/(Cs₀*L₀).

12. The method of claim 11 wherein selecting components for use in the filtering element based on the suitable values for the inductance and parasitic capacitance includes identifying particular components from among the candidate components having central inductance and parasitic capacitance values corresponding to the particular combinations of suitable L₀ and Cs₀ values.

13. The method of claim 1 wherein determining suitable values for inductance and parasitic capacitance sufficient to achieve a target impedance value at a selected RF signal frequency based on the tolerances for the inductances and parasitic capacitances of the candidate components additionally exploits a parasitic resistance (Rs) of the inductive element.

14. The method of claim 13 wherein selecting components for use in the filtering element based on the suitable values for the inductance and parasitic capacitance includes selecting components have a parasitic resistance (Rs) less 75 ohms.

15. The method of claim 13 wherein determining suitable values for inductance and parasitic capacitance while accounting for the parasitic resistance (Rs) of the inductive element includes:
for a given L₀, determining a range of values for Cs₀ based on:
Cs₀²<(1+Q_{L}²(1−ΔCs/Cs₀)²)/[ω₀²Z₀²*(Q_{L}²*(ΔCS/CS₀+ΔCs/Cs₀*ΔL/L₀+ΔL/L₀)²+(1+ΔCs/Cs₀)²)
where ω₀=1/sqrt(L₀Cs₀), Q_{L}=ω₀L₀/Rs and with a tolerance of ΔCs/Cs₀ and ΔL/L₀.

16. An inductive (L) element designed using the method of claim 1.

17. The inductive (L) element of claim 16 wherein the inductive element is an inductor.

18. The inductive (L) element of claim 16 wherein the inductive element is part of an LCR network.

19. A lead for use with an implantable medical device subject to radio-frequency (RF) fields, the lead comprising:
an electrode;
a conductor connected to the electrode; and
an inductive filtering element connected along the conductor, wherein the filtering element includes an inductor achieving a target impedance at a particular frequency to be filtered, the inductor having an inductance and a parasitic capacitance, and wherein the inductance and parasitic capacitance are sufficient to achieve a target impedance value at the particular frequency despite variations in the inductance and parasitic capacitance due to device tolerance.

20. A method for designing a lead for use with an implantable medical device, wherein the lead includes an inductive filtering element to reduce lead heating due to radio-frequency (RF) fields, the inductive filtering element having an inductance and a parasitic capacitance, the method comprising:
selecting a self-resonant frequency (SRF) between at least two separate RF signal frequencies of a magnetic resonance imaging (MRI) system and selecting a target impedance to be achieved at each of the selected frequencies;
determining suitable values for inductance and parasitic capacitance sufficient to achieve the target impedance at each of the separate frequencies; and
selecting and installing particular components for use in the inductive filtering element based, in part, on the suitable values for inductance and parasitic capacitance.

21. The method of claim 20 wherein the separate frequencies include a first frequency in the range of 63.7±0.345 MHz and a second frequency in the range of 127.6±3.6 MHz.

22. The method of claim 20 wherein determining suitable values for inductance and parasitic capacitance sufficient to achieve the target impedance at each of the separate frequencies includes:
selecting an inductance for the inductive filtering element and then determining a range of suitable parasitic capacitance values sufficient to achieve the target impedance at each of the separate frequencies.

22. The method of claim 21 wherein determining a range of suitable parasitic capacitance values sufficient to achieve the target impedance at both of a pair of MRI frequencies includes:
separately solving the following equation for parasitic capacitance (Cs) for each of the two frequencies of the pair of frequencies:
Z²=(1+Q_{L}²)/[(YS−Q_{L}ω₀Cs)²+ω₀²Cs²]
where Z is the target impedance, ω₀ is representative of the frequency, Q_{L} represents a resonance factor for the inductor and Ys represents the reciprocal of the parasitic resistance (Rs) of the inductor; and
determining the range of parasitic capacitance values from the solutions to the equations.

23. The method of claim 22 wherein determining a range of suitable parasitic capacitance values sufficient to achieve the target impedance at both of the MRI frequencies further includes determining the range of suitable parasitic capacitance values based on tolerances in the inductance and parasitic capacitance values of candidate inductive elements.

24. An inductive (L) element designed using the method of claim 20.

25. The inductive (L) element of claim 24 wherein the inductive element is an inductor.

26. The inductive (L) element of claim 24 wherein the inductive element is part of an LCR network.

27. A lead for use with an implantable medical device subject to radio-frequency (RF) fields at a plurality of separate frequencies of a magnetic resonance imaging (MRI) system, the lead comprising:
an electrode;
a conductor connected to the electrode; and
an inductive filtering element connected along the conductor, wherein the filtering element includes an inductor achieving a target impedance at each of the plurality of separate RF signal frequencies, the inductor having an inductance and a parasitic capacitance providing a self-resonant frequency between the separate RF signal frequencies, and wherein the inductance and parasitic capacitance are sufficient to achieve the target impedance value at each of the separate RF signal frequencies.
